(19) United States
(12) Patent Application Publication
(10) Pub. No.: US 2020/0390052 A1
(43) Pub. Date: Dec. 17, 2020

(54) CHINESE LANTERN CLONAL LINE KI1496

(71) Applicant: KEMIN INDUSTRIES, INC., Des Moines, IA (US)

(72) Inventors: Brindha Narasimhamoorthy, West Des Moines, IA (US); John Andrew Greaves, Ankeny, IA (US); Liuqing Zhao, Zhuhai (CN); Zhiqiang Qiu, Qinzhou (CN)

(21) Appl. No.: 16/898,858

(22) Filed: Jun. 11, 2020

Related U.S. Application Data
(60) Provisional application No. 62/860,578, filed on Jun. 12, 2019.

(51) Int. Cl. A01H 6/82; A01H 5/10

(52) U.S. Cl. CPC A01H 6/82; A01H 5/10

(57) ABSTRACT

The present invention relates generally to a Chinese lantern plant and, more specifically, to a proprietary clonal plant line KI1496 that contains elevated amounts of zeaxanthin and β-cryptoxanthin.

### CROSS-REFERENCE TO RELATED APPLICATIONS

This application claims the benefit of priority to U.S. Provisional Patent Application No. 62/860,578, filed Jun. 12, 2019, entitled “CHINESE LANTERN (PHYSALIS ALKEKENGI) PLANT LINE DENOMINATED KI1496,” the entire disclosure of which is hereby incorporated by reference in its entirety.

### FIELD OF THE INVENTION

The present invention relates generally to a Chinese lantern (Physalis alkekengi) plant and, more specifically, to a proprietary clonal plant line denominated KI1496 that produces elevated levels of zeaxanthin and β-cryptoxanthin.

### BACKGROUND OF THE INVENTION

Chinese lantern (Physalis alkekengi) a traditional Chinese herb, is a hardy perennial plant grown commercially in the northern provinces of China. The berries of Chinese lantern are edible and are grown for making juice, while the sepals are used in traditional Chinese medicine for the treatment of different ailments. Chinese lantern belongs to the Solanaceae family. It is characterized by having a swollen balloon-like calyx that encloses the mature berry. In Physalis alkekengi, this structure is often referred to as a “Chinese lantern,” but in other species with a similar structure, it has also been more formally termed “inflated-calyx syndrome.” He C. and Saedler H. Heterotopic expression of MPF2 is the key to the evolution of the Chinese lantern of Physalis, a morphological novelty in Solanaceae. PNAS. 102(16): 5779-5784 SA-11-00282 (2005).

Zeaxanthin and lutein, two widely studied xanthophylls are well-known to protect against age-related macular degeneration (AMD) and age-related cataracts. Products with enriched lutein are currently available in the human dietary supplement market. Several plants containing high amounts of total zeaxanthin have been studied for their potential use. Weller P. and Breithaupt D E. 2003. Identification and quantification of zeaxanthin esters in plants using liquid chromatography-mass spectrometry. J. Agric. Food Chemistry. 51:7044-7049 SA-10-01286.

The commercial launch of a naturally-sourced zeaxanthin product has been considered challenging for quite some time given the difficulty in identifying a suitable source with sufficiently high levels of zeaxanthin to be economically feasible. Several plant species were studied for the presence of adequate amounts of zeaxanthin for the development of a commercial product. From those sources studied, dried berries of Chinese wolfberry (Lycium barbarum), Chinese lanterns (Physalis alkekengi), orange pepper (Capsicum annuum), and sea buckthorn (Hippophae rhamnoides) were found to contain the highest levels of zeaxanthin ester. Weller P. and Breithaupt D E. 2003. Identification and quantification of zeaxanthin esters in plants using liquid chromatography-mass spectrometry. J. Agric. Food Chemistry. 51:7044-7049 SA-10-01286.

One study reported that berries of Chinese lanterns grown in the conditions of the Belgorod area of Russia, accumulated combined zeaxanthin and β-cryptoxanthin derivatives at levels of up to 20 mg/g of husks. Deineka V I, Sorokopudov V N, Deineka L A, Tret'yakov MYu, Fesenko V V. 2008. Studies of Physalis alkekengi L. berries as a source of xanthophylls. Pharmaceutical Chemistry Journal. 42: 36-37. SA-11-00264. In addition, in the berries of Chinese lantern were found to have small amounts of zeaxanthin, about 0.3 mg/berry on average. Deineka V I, Sorokopudov V N, Deineka L A, Tret'yakov MYu, Fesenko V V. 2008. Studies of Physalis alkekengi L. berries as a source of xanthophylls. Pharmaceutical Chemistry Journal. 42: 36-37. SA-11-00264.

### SUMMARY OF THE INVENTION

The present invention relates to a plant or clonal line of Chinese lantern (Physalis alkekengi) plant and, more specifically, to a proprietary clonal plant line denominated KI1496 that has elevated levels of zeaxanthin and β-cryptoxanthin, excellent vigor and overall robust agronomic traits. The present invention was selected through a robust breeding program and has been asexually propagated to produce a clonal line of identical plants.

Plants of the clonal line KI1496 have not been observed under all possible environmental conditions. The phenotype may vary somewhat with variations in environment and culture such as temperature, light intensity, day length, water status, and/or fertilizer rate or type without, however, any variance in genotype.

Another object of the invention is a variety of Chinese lantern that is novel, stable, and uniform and has good agronomic characteristics that permit efficient cultivation of the variety as a crop that produces a high amount of biomass from which zeaxanthin can be extracted.

### DETAILED DESCRIPTION OF THE FIGURES

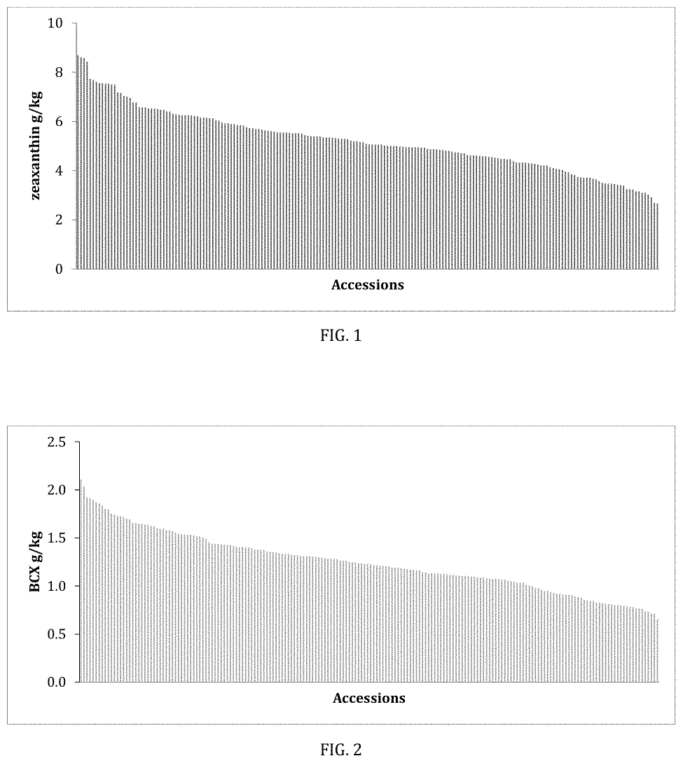

FIG. 1 depicts the zeaxanthin levels in g/kg in 190 Chinese lantern plants.

FIG. 2 depicts the β-cryptoxanthin levels in g/kg in freeze dried sepals in 190 Chinese lantern plants.

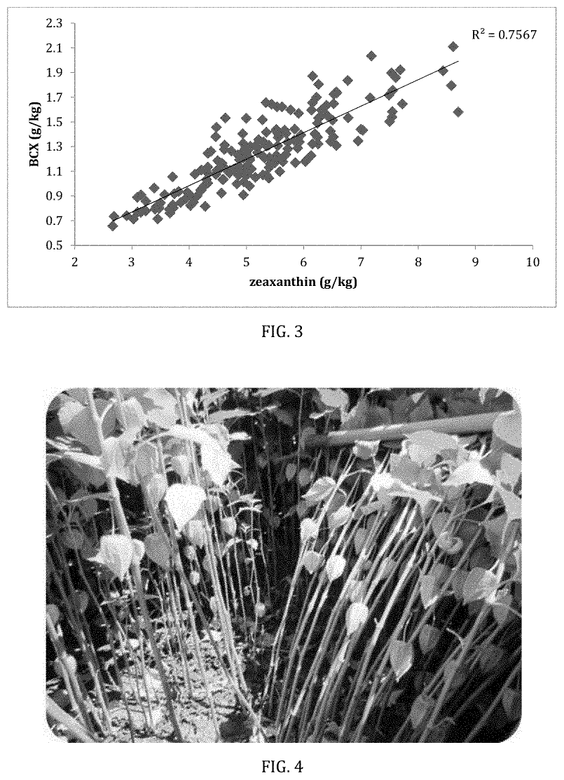

FIG. 3 shows the correlation between zeaxanthin content and β-cryptoxanthin content in the sepals.

FIG. 4 is a photograph showing Chinese lantern as it emerges as SVS during the 2nd year of growth.

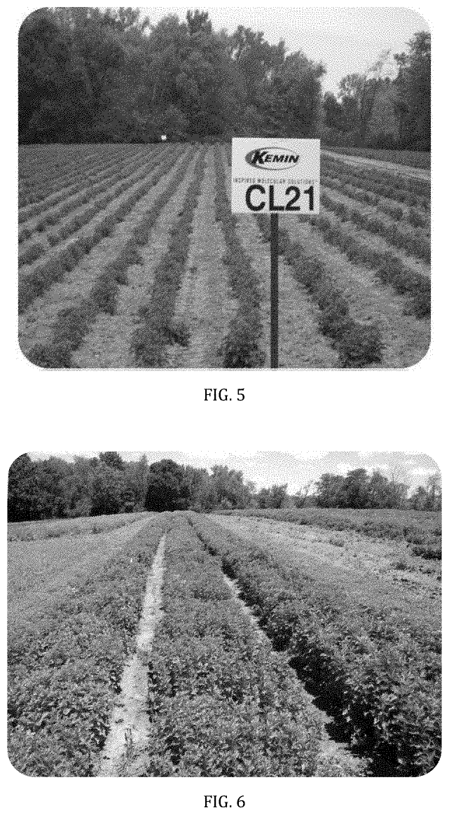

FIG. 5 is a photograph showing Chinese lantern individual plants in the 1st year of establishment from propagates.

FIG. 6 is a photograph showing Chinese lantern clonal lines plot at the vegetative stage.

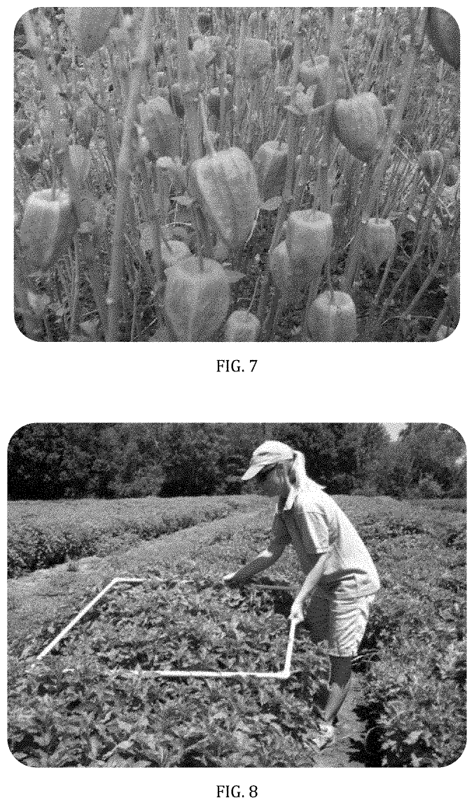

FIG. 7 is a photograph showing Chinese lantern clonal lines plot at the lantern maturity stage.

FIG. 8 is a photograph showing Chinese lantern at the 1 m² count.

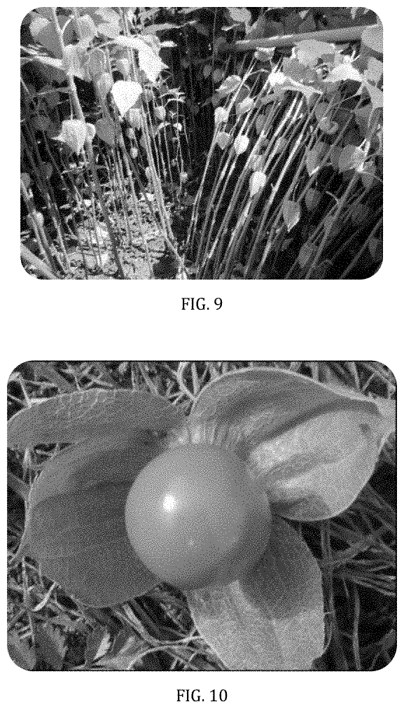

FIG. 9 is a photograph showing Chinese lantern at the SVS/m² count.

FIG. 10 is a photograph showing a fully mature lantern.

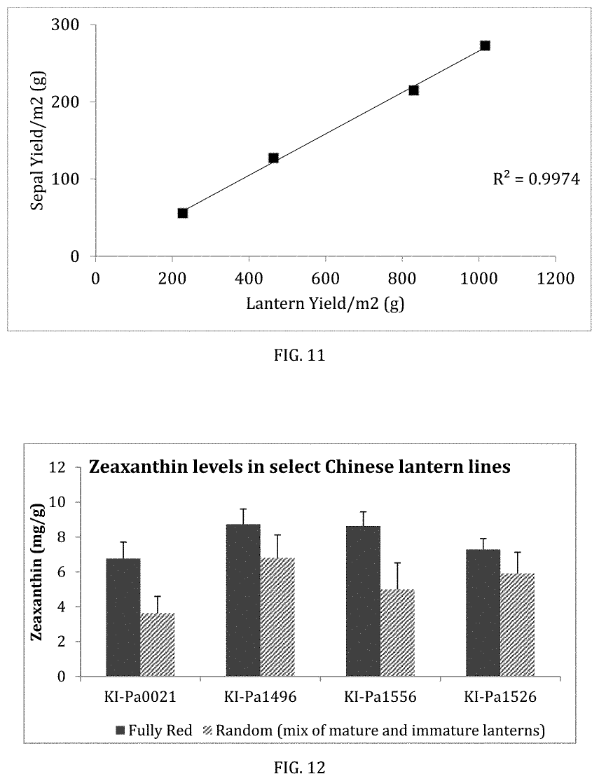

FIG. 11 shows the correlation between lantern yield and sepal yield in the Chinese lantern plant.

FIG. 12 shows the zeaxanthin levels in selected Chinese lantern plants.

### DETAILED DESCRIPTION OF THE INVENTION

The Chinese lantern clonal line KI1496 is a proprietary clonal line developed through selection.

Materials and Methods:

A total of five Chinese lantern accessions were purchased from medicine centers in China and investigated as potential sources of zeaxanthin and β-cryptoxanthin (BCX). The sepal samples tested in this study had the highest levels of zeaxanthin observed at Kemin so far at >5 g/kg; and BCX levels >1.2 g/kg. This study showed that these high carotenoid levels may be sufficient for Physalis alkekengi to be an economically viable commercial source of zeaxanthin and BCX for Kemin Health.

A rapid, high throughput method was developed to evaluate zeaxanthin and beta-cryptoxanthin (BCX) levels in dried Chinese lantern (Physalis alkekengi L.) sepals for genetic screening purposes. Sample preparation involved a modified AOAC (970.64) method in order to improve throughput. Dried Chinese lantern sepals were extracted and saponified with a four-solvent blend of hexane:ethanol:acetone:toluene 10:6:7:7 and 40% methanolic potassium hydroxide. Hot saponification in the AOAC method was replaced by ultrasonic saponification in order to scale down the sample and solvent amounts and to reduce sample preparation time. Diethyl ether was used to replace hexane as the extraction solvent in order to increase the solubility of zeaxanthin and BCX during the extraction process. A reverse-phase HPLC method was developed for the determination of zeaxanthin and BCX using a YMC carotenoid C30 column with a 15-minute gradient.

Simultaneously a genetic screening of 190 individuals in a Chinese lantern population was conducted for carotenoid levels and yield components. The seed of Chinese lantern obtained from an independent commercial source (New England Seeds, Hartford Conn.) were planted at Kemin's greenhouse in Iowa, USA. A total of 200 random seedlings from the population were transplanted into 5-gallon pots during May 2010 of which 199 survived. The entire fruiting structures known as lanterns are comprised of bright orange sepals fused together enclosing small round berries within. Once the lanterns turned bright orange, they were considered as mature and ready for collection (September, 2010). One to five representative lanterns, depending upon availability, were collected per individual plant and care was taken to make sure that all were at the same stage of maturity on a visual basis. Berries inside the lanterns were removed and the sepals of the lanterns were freeze dried. Sepal samples from individual plants were sent for carotenoid analysis. The total number of lanterns for each individual plant was counted.

An additional one to three lanterns at the same maturity stage were harvested to obtain the fresh weights. Berries were removed to obtain the berry weight and sepal weight separately. From this data, average lantern weight, sepal weight and single berry weight were calculated. Total lantern yield on wet weight basis for each plant was obtained by multiplying total number of lanterns by average lantern weight.

Analysis:

Conventional analytical methods were used to determine the content of the zeaxanthin and BCX in dried Chinese lantern sepals. The method was validated for standard linearity, range, robustness, precision, accuracy, stability, limit of detection and limit of quantitation. Dried Chinese lantern sepals were ground to a powder and were extracted with a HEAT solvent mix (hexane:ethanol:acetone:toluene) and methanolic potassium hydroxide. The samples were analyzed on an Agilent HPLC; and zeaxanthin and BCX were expressed in mg/g on dry matter basis.

There was considerable variation observed for zeaxanthin and BCX content; number of lanterns per plant and total lantern yield. A strong linear relationship between zeaxanthin and BCX levels suggested a tight linkage between the two molecules requiring a mutation approach to select for each molecule independently. Lantern number per plant was identified as the major component of total lantern yield. Five individual clonal lines designated as KI-Pa0001, KI-Pa0021, KI-Pa0022, KI-Pa0029, KI-Pa0035 and KI-Pa0178 had the highest carotenoid levels combined with total lantern yield and were selected for larger scale field evaluation. The means ranges and standard deviation (SD) for both zeaxanthin and BCX levels are given in Table 1. Several individuals with zeaxanthin levels >8 mg/g and BCX levels >2 mg/g were identified. The researched observed a wide range of variation for both sepal zeaxanthin content (Table 1; FIG. 1) and sepal BCX content (Table 1; FIG. 2) among the individual plants tested.

TABLE 1
Mean, range and SD for zeaxanthin and β-cryptoxanthin
among 191 Chinese lantern genotypes

| | Zeaxanthin levels (g/kg) | β-cryptoxanthin levels (g/kg) |
|---|---|---|
| Range | 2.66-8.79 | 0.66-2.11 |
| Average | 5.21 | 1.24 |
| SD | 1.232 | 0.304 |

The researchers further observed that zeaxanthin content was positively correlated to BCX content in sepals (FIG. 3).

The studies done in 2010 showed that Chinese lantern (Physalis alkekengi L.) is an economical and agronomically scalable source of zeaxanthin and beta-cryptoxanthin (BCX) to expand Kemin carotenoid product platform. A follow-up study was conducted to compare the levels of these two molecules in chili pepper and Chinese wolfberry. Chili pepper is a crop which is widely grown in the southwest region of the USA while Chinese wolfberry is grown in limited acreages in China.

Chili Pepper:

A total of 112 pepper varieties were grown outdoors in pots to produce fruit for analysis. The fruit was harvested between August and November. The fruit was freeze-dried and used for analysis.

Chinese Wolfberry:

Fruit of 13 Chinese wolfberry varieties were obtained from the National Wolfberry Research Center in China and two samples were obtained from the United States. The fruit were freeze-dried for analysis.

Chemotyping:

Carotenoids were extracted and quantitated using conventional HPLC methods.

Chili Pepper:

The zeaxanthin levels in peppers ranged from near 0 to 0.72 g/kg by dry weight, as shown in Table 2. Nine varieties had no detectable zeaxanthin at any harvest. Levels of BCX in peppers ranged from near 0 to 0.027 g/kg by dry weight. Thirty-two of the pepper varieties had no detectable BCX. The commercial pepper variety “Pasilla” had the highest concentration of both molecules at 0.72 g/kg (Zeaxanthin) and 0.27 g/kg (BCX), as shown in Table 3.

Chinese Wolfberry:

The highest level of zeaxanthin present in Chinese wolfberry was 0.95 g/kg (Table 3). The highest level of BCX was 0.041 g/kg in accession NWRC-03 which also had second highest level of zeaxanthin, as shown in Table 3.

The Chinese lantern showed the highest levels of zeaxanthin and BCX content compared to Chili pepper and Chinese wolfberry.

TABLE 2
Mean Zeaxanthin and β-cryptoxanthin (BCX) concentrations

| | No. of | Zeaxanthin (g/kg) | | | BCX (g/kg) | | |
|---|---|---|---|---|---|---|---|
| Crop | samples | Range | Mean | SD | Range | Mean | SD |
| Chinese | 190 | 2.66-8.79 | 5.21 | 1.24 | 0.66-2.11 | 1.24 | 0.30 |
| lantern | | | | | | | |
| Chinese | 15 | 0.065-0.91 | 0.48 | 0.24 | 0.013- | 0.023 | 0.006 |
| Wolfberry | | | | | | | |
| Chili | 243 | ND-0.72 | 0.11 | 0.09 | ND-0.27 | 0.023 | 0.033 |
| pepper | | | | | | | |

TABLE 3
Concentrations of Zeaxanthin and β-cryptoxanthin
(BCX) in the five best Chili Peppers, Chinese wolfberry
lines as compared to Chinese lantern clonal lines.

| | | Zeaxanthin | BCX |
|---|---|---|---|
| Crop | Entry | (g/kg) | (g/kg) |
| Pepper | Pasilla | 0.72 | 0.270 |
| Pepper | Goat Horn | 0.57 | 0.140 |
| Pepper | Purple Serrano | 0.37 | 0.079 |
| Pepper | Mulatto | 0.37 | 0.087 |
| Pepper | Numex Garnet | 0.30 | 0.147 |
| Pepper | Numex 641 | 0.25 | 0.130 |
| Pepper | Numex Heritage | 0.12 | 0.190 |
| | Big Jim | | |
| Chinese | Virginia-fresh | 0.95 | 0.023 |
| wolfberry | | | |
| Chinese | NWRC-04 | 0.91 | 0.041 |
| wolfberry | | | |
| Chinese | NWRC-03 | 0.68 | 0.025 |
| wolfberry | | | |
| Chinese | NWRC-02 | 0.60 | 0.021 |
| wolfberry | | | |
| Chinese | NWRC-05 | 0.60 | 0.027 |
| wolfberry | | | |
| Chinese | NWRC-09 | 0.48 | 0.023 |
| wolfberry | | | |
| Chinese lantern | KI-Pa0001 | 8.58 | 1.79 |
| | KI-Pa0021 | 7.69 | 1.92 |
| | KI-Pa0022 | 7.18 | 2.03 |
| | KI-Pa0029 | 7.53 | 1.91 |
| | KI-Pa0035 | 8.70 | 1.58 |
| | KI-Pa0035 | 8.61 | 2.11 |

Conclusion:

In summary, the Chinese lantern clonal lines had considerably higher zeaxanthin and BCX levels compared to other tested varieties, including chili pepper and Chinese wolfberry varieties. Notably, concentrations of zeaxanthin and BCX in the even the best chili pepper and Chinese wolfberry varieties were still significantly lower than many of the Chinese lantern lines evaluated.

Selective Breeding Program:

Based on the conclusion that Chinese lantern was the best source of zeaxanthin, a large scale genetic screening was done to identify potential Chinese lantern clonal lines that were suitable for commercial production. A collection of Chinese lantern seed from publicly available sources were evaluated (Burpee Seeds, Richter's Seeds, Thompson and Morgan Seeds, Ritcher's Seeds, myseedneeds.com, rareexoticseeds.com). A total of 1700 individual plants were grown from 13 distinct Chinese lantern seed sources from May to October in Des Moines, Iowa. When mature, 5-6 representative lanterns, depending upon availability, were collected per individual plant and care was taken to make sure that all were at the same stage of maturity on a visual basis. Berries inside the lanterns were removed and the sepals of the lanterns were freeze dried for carotenoid analysis. Lantern numbers from each plant was counted and recorded.

HPLC assay was used for the screening and relative quantitation of zeaxanthin and BCX molecules in these individuals. The individual segregating plants within this population were scored on a qualitative basis and visually for number of lanterns per plant and lantern size. There were several dozen clonal lines with zeaxanthin levels >5 g/kg. and 14 clonal lines with zeaxanthin levels >7 g/kg were identified (Table 4). The top five clonal lines (KI-Pa1563, KI-Pa1526, P-86, KI-Pa1496, KI-Pa1556, KI-Pa1554) with zeaxanthin levels >9 g/kg were selected.

TABLE 4
Concentrations of Zeaxanthin and β-cryptoxanthin
(BCX) in top 14 Chinese lantern lines identified

| | Zeaxanthin (g/kg) | BCX (g/kg) |
|---|---|---|
| KI-Pa1563 | 9.96 | 1.75 |
| KI-Pa1526 | 9.94 | 2.01 |
| P-86 | 9.70 | 1.93 |
| KI-Pa1556 | 9.44 | 2.19 |
| KI-Pa1496 | 9.42 | 2.10 |
| KI-Pa1554 | 9.02 | 1.94 |
| KI-Pa1541 | 8.42 | 1.41 |
| KI-Pa1600 | 8.29 | 1.73 |
| KI-Pa1511 | 8.07 | 1.63 |
| KI-Pa1500 | 7.83 | 1.51 |
| KI-Pa1546 | 7.83 | 1.61 |
| KI-Pa1524 | 7.66 | 1.65 |
| KI-Pa0471 | 7.60 | 1.38 |
| P-101 | 7.01 | 1.35 |
| KI-Pa0021 | 6.40 | 1.92 |

From these clonal lines, three clonal lines (KI-Pa1496, KI-Pa1556 and P-86) with zeaxanthin levels >9.4 g/kg combined with higher lantern numbers and one clonal line (P-101) with highest number of lantern were propagated in greenhouses in Iowa and Texas for rhizome production during winter months of 2011-2012. During the following spring, these lines were planted in long rows alongside KI-Pa0021 (previously identified clonal line) at Kemin's field location in Iowa for carotenoid and phenotype evaluation. The remaining selections were planted in small plots at the same field location for comparison. Simultaneously, these selections were clonally propagated and planted as propagates.

Chinese lantern planted as individually propagated plants produce a mat of rhizomes underground during the first year of establishment. When rhizomes are dug and planted, shoots emerge from the rhizome filling in the area slowly. Chinese lantern plants are highly “plastic,” changing morphology from the first year to the second. Plants emerge in the second year onwards from each node of the subterranean rhizomes as single vertical stems (SVS) (FIGS. 4, 5). Each SVS produces 1 to 5 lanterns. Plants remain in rows during the first year of establishment; and spread into a meadow during the second year of expansion. The plant phenotype changes from single bushy plants the first year, to many single vertical stems (SVS) in the second year and beyond (FIGS. 4, 5). Lantern yield is lower during the first year, while maximum yield can be realized from the second year onwards. Hence, any agronomic evaluation of new Chinese lantern clonal lines is only appropriate if done during the second year of growth.

For zeaxanthin extraction, lanterns were harvested at the time of lantern maturity. Lantern maturity is usually assessed visually when sepals turn from green to red in color. The color change from green to red, referred to as ‘ripening’, occurs over an extended period where it takes about 20-30 days from the time of partial color change to a complete color change (FIGS. 6, 7). The color change from green to red begins at the top of the lantern and progresses until the sepals and berries turn completely red. Maturity of the whole lantern fruit is one of the major factors that determine the carotenoid composition of the sepals. When harvested at the right maturity stage, lanterns seem to have the maximum carotenoid accumulation in the sepal.

Quantitative data were collected from each of the experimental clonal lines for the traits as described below from a replicated, defined unit area of 1 m² (FIG. 8). For each experimental clonal line, three 1 m² areas representing three replicates were chosen within each plot. The three replicates for each clonal line were chosen within the plot in such a way that they would represent the plot. The plot size for the experimental clonal line varied from 4 m² to 8 m² depending upon the availability of propagules at the time of planting. Maturity related traits were based on the entire plot and the data was un-replicated.

Number of SVS/m²:

Total number of single vertical stems was counted from each clonal line from 1 m² unit area to obtain number of SVS per m².

Number of Lanterns/SVS:

Total number of lanterns were counted from 20 SVS within the 1 m² unit area to obtain average number of lanterns/SVS.

Number of Lanterns/m²:

Total number of lanterns was counted from 1 m² area for each experimental clonal line to obtain average lantern number per unit area.

Svs Height:

SVS height was calculated as the mean distance from the soil level to the tips of a random handful of SVS, averaged across three replicates for each of the experimental clonal lines.

Maturity:

Maturity was assessed by two main criteria: uniformity of color change from green to red; and earliness to maturity. Each experimental clonal line was monitored every week for the uniformity of color change on a visual basis during August, 2012.

Lantern Yield/m²:

The lanterns from all the SVS within the 1 m² area were harvested and weighed for each experimental clonal line.

Sepal and Berry Yield/m²:

Sepals were separated from berries in approximately 50 lanterns of each experimental clonal line to obtain relative sepal weight and berry weight. Data was taken from three replicates. The total lantern weight, sepal and berry weights were used to obtain berry yield/m² and sepal yield/m² for each clonal line.

Berry:Sepal Ratio:

Berry yield/m² and sepal yield/m² was used to obtain the berry:sepal ratio for each clonal line.

Sepal and Berry Yield/Acre:

The projected sepal yield/acre was calculated based on data from the m² area.

Although data was taken on number of SVS/m² and lantern/SVS, these two traits did not correlate with lantern yield. The researchers observed that clonal lines could have more stems per unit area but may or may not bear lanterns. Therefore, lanterns/m² was more of an appropriate yield component and estimate. Both KI-Pa1496 and KI-Pa1526 produced more lanterns/m² than other lines. Thus, an increase in SVS did not correlate with an increase in lantern set or lantern yield.

Lantern yield/m² most accurately estimated weighing all lanterns in 1 m², in which both KI-Pa1496 and KI-Pa1526 developed significantly more lanterns/m² as shown in Table 5. Both KI-Pa1496 and KI-Pa1526 showed significantly higher sepal and lantern yield, as shown in Table 6. The researchers observed a strong correlation between lantern and sepal yield (FIG. 11, R2=0.9974).

TABLE 5
Lantern yield from 1 m² area and per acre basis in year 1 (Iowa)

| Clonal line | Average (g) | Projected Lantern yield/acre (kg) |
|---|---|---|
| KI-Pa0021 | 464 ± 392 | 1877.8 |
| KI-Pa1496 | 830 ± 222 | 3360.3 |
| KI-Pa1526 | 1016 ± 256 | 4111.7 |
| KI-Pa1556 | 227 ± 27 | 918.6 |

TABLE 6
Sepal and berry yield data in year 1 (Iowa)

| | | | | Lantern | Sepal | Berry |
|---|---|---|---|---|---|---|
| | Sepal | Berry | | Yield/ | Yield/ | Yield/ |
| Clonal | yield/ | yield/ | Berry:Sepal | Acre | Acre | Acre |
| line | m²(g) | m²(g) | ratio | (kg) | (kg) | (kg) |
| KI-Pa0021 | 127.3 | 324.6 | 2.6:1 | 1877.8 | 521 | 1356.1 |
| KI-Pa1496 | 214.7 | 610.4 | 2.8:1 | 3360.3 | 884.5 | 2475.8 |
| KI-Pa1526 | 272.7 | 743.3 | 2.7:1 | 4111.7 | 1111.3 | 3000.5 |
| KI-Pa1556 | 55.9 | 170.3 | 3.1:1 | 918.6 | 224 | 694.5 |

Lantern yield in KI-Pa1496 doubled from year two onwards and projected lantern yield per acre was estimated at >8000 kg/acre. KI-Pa1496 set lanterns that matured earlier than other lines with >90% of lanterns turning red by early September. Zeaxanthin levels in KI1496 in comparison to other lines is shown in FIG. 12.

KI-Pa1496 had significantly higher zeaxanthin accumulation compared to all lines for multiple years tested. KI-Pa1496 had significantly higher yield over many other selected lines at the Kemin research farm in Iowa. KI-Pa1496 had the earliest and most uniform maturity in ripening of lanterns of all lines, as well as the most vigorous in overall plant health and lantern set.

Extraction Methods:

Zeaxanthin and BCX are both common xanthophylls, a class of carotenoid alcohols. Zeaxanthin and lutein have the same molecular weight but not the same chemical structure. Consequently, they are isomers but not stereoisomers. The only difference is the location of the double bond in one of the 6-member rings. BCX is closely related to zeaxanthin by its chemical structure, at the exception of the lack of one hydroxyl group on one of the 6-member ring. They will have related but not identical chemical properties. Accordingly, persons of ordinary skill in the art would appreciate that the extraction properties of zeaxanthin and BCX will be relatively similar to the one of lutein, and known methods and extraction techniques would fall within the scope of the present disclosure. By way of non-limiting example, U.S. Pat. Nos. 5,648,564; 6,797,303; and 6,818,239 describe known extraction methods and are incorporated in their entirety herein. Furthermore, lutein, zeaxanthin and BCX are present under an ester form within the plant material. Persons of ordinary skill in the art would readily appreciate that the zeaxanthin and BCX can be esterified by fatty acids, such as lauric, myristic and palmitic acids.

Hexane has been a common organic solvent to extract the ester form of xanthophylls from plant material, and has been known for decades. In addition to hexane, other straight chain hydrocarbons such as pentane, heptane, or petroleum ether (b.p.=30° C.-60° C.) can be effectively used as solvents. Indeed, there are many other organic solvents that have been used to extract carotenoids from plants, including but are not limited to methanol, acetone, ethyl acetate, diethyl ether, petroleum ether, chloroform, tetrahydrofuran and supercritical carbon dioxide. Persons of ordinary skill in the art would readily recognize other solvents suitable for the extraction of zeaxanthin and/or BCX.

Following extraction, the xanthophyll esters can be saponified in the presence of a base in solution. The most common bases employed are sodium hydroxide and potassium hydroxide. Any other alkali metal hydroxides can be used for this hydrolysis. Propylene glycol is commonly used to solubilize the base. Other solvents such as water and organic alcohols can be used. Organic alcohols include but are not limited to ethanol, 1-butanol, 2-butanol, 2-methyl-1-propanol, 1-propanol, and 2-propanol, which are all listed as safe in Class 3 solvents by the FDA. Preferred solvents are also selected for their boiling points. Solvents with boiling points of about 75° C. to 120° C. are preferred.

According to certain embodiments of the present invention, the Chinese Lantern extract can be a liquid or dry product that contains zeaxanthin and/or BCX.

According to at least one embodiment of the present invention, the Chinese Lantern extract is incorporated into human food, for instance incorporated into the food itself or applied to the surface of the food, wherein the extract contains zeaxanthin and/or BCX.

According to at least one embodiment of the present invention, the Chinese Lantern extract is incorporated into animal food, including for instance, pet food or animal feed. By way of non-limiting example, the extract can be incorporated into the food itself or applied to the surface of the food, wherein the extract contains zeaxanthin and/or BCX.

According to at least one embodiment of the present invention, the Chinese Lantern extract is incorporated into a dietary supplement, wherein the extract contains zeaxanthin and/or BCX. As used herein, dietary supplement refers to a product taken orally that contains one or more ingredients, such as zeaxanthin and/or BCX, that is intended to supplement a human or animal diet. Non-limiting examples of dietary supplements include products that are orally administered, such as capsules, softgels, tablets, powders or liquid products.

According to at least one embodiment of the present invention, the Chinese Lantern extract is incorporated into a beverage, wherein the extract contains zeaxanthin and/or BCX. For instance, the extract may be a liquid or dry product that is incorporated into a beverage. As used herein, beverage refers to a drinkable liquid. Non-limiting examples of beverages include carbonated or uncarbonated products, flavored water products, or liquid shakes.

According to at least one embodiment of the present invention, the Chinese Lantern extract is incorporated into a personal care product, wherein the extract contains zeaxanthin and/or BCX. Non-limiting examples of personal care products include topical applications, such as lotions, creams, oils, or suspensions.

Certificate of Deposit

The Chinese lantern plant denominated KI1496 was produced by a proprietary plant line deposited under the terms of the Budapest Treaty with the ATCC on Mar. 14, 2019 and assigned accession number PTA-125794.

Evidence of Uniformity and Stability

No variants of any kind have been observed since the variety KI1496 was identified, indicating the stability and uniformity of the genotype. It is clear from these results that the KI1496 cultivar is stable and reproduces true to type in successive generations of asexual reproduction.

Statement of Distinction

KI1496 consistently produces higher per dry weight levels of zeaxanthin as compared to other selected and commercial varieties while its vigor and biomass is equivalent to other commercial varieties.

Having described the invention with reference to particular compositions, theories of effectiveness, and the like, it will be apparent to those of skill in the art that it is not intended that the invention be limited by such illustrative embodiments or mechanisms, and that modifications can be made without departing from the scope or spirit of the invention, as defined by the appended claims. It is intended that all such obvious modifications and variations be included within the scope of the present invention as defined in the appended claims. The claims are meant to cover the claimed components and steps in any sequence which is effective to meet the objectives there intended, unless the context specifically indicates to the contrary.

The foregoing description has been presented for the purposes of illustration and description. It is not intended to be an exhaustive list or limit the invention to the precise forms disclosed. It is contemplated that other alternative processes and methods obvious to those skilled in the art are considered included in the invention. The description is merely examples of embodiments. It is understood that any other modifications, substitutions, and/or additions may be made, which are within the intended spirit and scope of the disclosure. From the foregoing, it can be seen that the exemplary aspects of the disclosure accomplishes at least all of the intended objectives.

1. A method of producing zeaxanthin and/or BCX, comprising extracting zeaxanthin and/or BCX from plant tissue of a Chinese lantern plant denominated KI1496 as produced by a plant line deposited with the ATCC and assigned accession number PTA-125794.

2. The method of claim 1, wherein the plant tissue is selected from the group consisting of berries, sepals, lanterns, leaf, rhizome, root, seed, or stem tissue.

3. The method of claim 1, further comprising using the extracted zeaxanthin and/or BCX as an antioxidant in a human food, animal food, dietary supplement, beverage or personal care product.

4. A method of providing a carotenoid, comprising extracting zeaxanthin and/or BCX from plant tissue of a Chinese lantern plant denominated KI1496 as produced by a plant line deposited with the ATCC and assigned accession number PTA-125794.

5. The method of claim 4, wherein the carotenoid is incorporated into a human food, animal food, dietary supplement, beverage or personal care product.

6. A method of making a carotenoid-containing product, comprising:
a. growing a Chinese lantern plant denominated KI1496 as produced by a plant line deposited with the ATCC and assigned accession number PTA-125794;
b. harvesting the mature lanterns;
c. extracting zeaxanthin and/or BCX from the mature lanterns; and
d. including the extracted zeaxanthin and/or BCX in a carotenoid-containing product.

7. The method of claim 6, wherein the tissue is selected from the group consisting of berries, sepals, lanterns, leaf, rhizome, root, seed, or stem tissue.

8. The method of claim 6, wherein the carotenoid-containing product is selected from the group consisting of human food, animal food, dietary supplements, beverages and personal care products.

9. A method by which the Chinese lantern plant line denominated KI1496 is used in a breeding program by using pollen from said clonal lines as a male parent; or flowers of said clonal lines as a female parent, for the generation of seed of a breeding population from which further selections can be made.

10. A plant or node of the Chinese lantern plant line denominated KI1496, or a cutting or part thereof, wherein representative plant tissue of KI1496 has been deposited with the ATCC and assigned accession number PTA-125794.

11. The plant of claim 10, wherein the plant or part thereof includes a leaf, rhizome, root, seed, or stem tissue.

12. The plant or node of claim 10, wherein the plant, node, cutting or part of the plant are used as a starting material to grow additional plants.

13. Pollen of the plant of claim 10.

14. An ovule of the plant of claim 10.

15. A method for producing Chinese lantern seed, said method comprising the steps of: (i) growing one or more plants from one or more of the plant or node of claim 10; (ii) pollinating one or more plants produced from one or more of the node or plant of claim 10 by self-pollination or by pollination with pollen from a different plant and, (iii) harvesting the resultant seed and growing a new population of plants.
